(19) United States
(12) Patent Application Publication
(10) Pub. No.: US 2012/0143121 A1
(43) Pub. Date: Jun. 7, 2012

(54) CHITOSAN SPREADING SYSTEM USING LOW TEMPERATURE ATMOSPHERIC PRESSURE PLASMA

(75) Inventors: Yark Yeon Kim, Daejeon (KR); Han Young Yu, Daejeon (KR); Yong Ju Yun, Daejeon (KR); Won Ick Jang, Daejeon (KR)

(73) Assignee: Electronics and Telecommunications Research Institute, Daejeon (KR)

(21) Appl. No.: 13/302,300

(22) Filed: Nov. 22, 2011

(30) Foreign Application Priority Data
Dec. 7, 2010 (KR) 10-2010-0124441

(51) Int. Cl. A61M 37/00

(52) U.S. Cl. 604/24

(57) ABSTRACT

Provided is a chitosan spreading system using low temperature atmospheric pressure plasma. The system includes a dielectric tube of hollow cylindrical shape including a gas inlet supplied with a carrier gas and a plasma outlet spraying low temperature atmospheric pressure plasma generated therein, a first electrode provided in the dielectric tube, an power supply unit configured to apply an electric power to the first electrode, a carrier gas supply unit configured to supply a carrier gas into the gas inlet of the dielectric tube, and a chitosan supply unit configured to supply chitosan into the low temperature atmospheric pressure plasma generated in the dielectric tube

### CROSS-REFERENCE TO RELATED APPLICATIONS

This U.S. non-provisional patent application claims priority under 35 U.S.C. §119 to Korean Patent Application No. 10-2010-0124441, filed on Dec. 7, 2010, in the Korean Intellectual Property Office, the entire contents of which are hereby incorporated by reference.

### BACKGROUND OF THE INVENTION

Embodiments of the inventive concepts relate generally to a chitosan spreading system. More particularly, embodiments of the inventive concepts relate to a chitosan spreading system configured to spread chitosan over skin using a low-temperature atmospheric pressure plasma.

Sterilization, blood coagulation, and cell regeneration are important factors in dressing or healing a variety of skin diseases or wounds, such as a burn, a diabetic foot ulcer, a venous foot ulcer, a decubitus ulcer, or a surgical wound. A skin diseases treatment apparatus should be developed to meet the demands for sterilization, blood coagulation, and cell regeneration.

Chitosan is a kind of amino-polysaccharide obtained by deacetylating chitin existent in nature (such as, a shell of crab or lobster, a cuttlebone, or a cell wall of mold, mushroom mycelium, or microbe). Furthermore, chitosan has an excellent bioaffinity in that it is non-toxic and biodegradable. In this sense, research relating to chitosan has been carried out in the fields of food, clothing, medicine, and health care. In particular, research is being extensively carried out to obtain desirable antibiotic, blood clotting, and cell regenerating properties of chitosan.

Good biological properties of chitosan (such as, antibiosis, blood coagulation, and cell differentiation and growth) are very sensitive to environmental temperature. For instance, at about 50° C. or more, such biological properties of chitosan may be abruptly deteriorated. In addition, a coating apparatus of chitosan should be able to provide a good adhesion property and a uniform coating property in order that chitosan can be utilized to heal a skin disease or wound.

Until the early 2000s, atmospheric pressure plasma had been studied to utilize a thermal property of plasma associated with surgical operations, such as blood coagulation or tissue removal, but a bacteria sterilization property of plasma has been widely applied in the fields of an air cleaner or a gas filter since the early 2000s. Recently, new medical apparatus is being actively studied on the basis of an interaction between plasma and biological cells. In particular, medical apparatuses with enhanced sterilization and cell regeneration capabilities are being developed based on an electrochemical property of plasma rather than the thermal property.

### SUMMARY

Embodiments of the inventive concepts provide a chitosan spreading system capable of improving absorption and adhesion properties of chitosan on a skin of a biological object.

According to example embodiments of the inventive concepts, a chitosan spreading system may include a dielectric tube of hollow cylindrical shape comprising a gas inlet supplied with a carrier gas and a plasma outlet spraying low temperature atmospheric pressure plasma generated therein, a first electrode provided in the dielectric tube, an power supply unit configured to apply an electric power to the first electrode, a carrier gas supply unit configured to supply a carrier gas into the gas inlet of the dielectric tube, and a chitosan supply unit configured to supply chitosan into the low temperature atmospheric pressure plasma generated in the dielectric tube.

According to other example embodiments of the inventive concepts, a chitosan spreading system may include at least one plasma generating device comprising a first dielectric tube of hollow cylindrical shape comprising a gas inlet supplied with a carrier gas and a plasma outlet spraying low temperature atmospheric pressure plasma generated therein, a first electrode provided in the first dielectric tube, an power supply unit configured to apply an electric power to the first electrode, and a carrier gas supply unit configured to supply a carrier gas into the gas inlet of the first dielectric tube, and a chitosan supplying device comprising a second dielectric tube of hollow cylindrical shape configured to supply chitosan into the low temperature atmospheric pressure plasma generated in the first dielectric tube.

### BRIEF DESCRIPTION OF THE DRAWINGS

Example embodiments will be more clearly understood from the following brief description taken in conjunction with the accompanying drawings. The accompanying drawings represent non-limiting, example embodiments as described herein.

FIGS. 1A through 1D are sectional views illustrating a chitosan spreading system according to example embodiments of the inventive concepts;

FIGS. 2A and 2B are sectional views illustrating a chitosan spreading system according to other example embodiments of the inventive concepts;

FIGS. 3A and 3B are sectional views illustrating a chitosan spreading system according to still other example embodiments of the inventive concepts;

FIGS. 4A and 4B are sectional views illustrating a chitosan spreading system according to even other example embodiments of the inventive concepts;

FIGS. 5A and 5B are sectional views illustrating a chitosan spreading system according to yet other example embodiments of the inventive concepts; and

FIG. 6 is a sectional view illustrating a chitosan spreading system according to further example embodiments of the inventive concepts.

It should be noted that these figures are intended to illustrate the general characteristics of methods, structure and/or materials utilized in certain example embodiments and to supplement the written description provided below. These drawings are not, however, to scale and may not precisely reflect the precise structural or performance characteristics of any given embodiment, and should not be interpreted as defining or limiting the range of values or properties encompassed by example embodiments. For example, the relative thicknesses and positioning of molecules, layers, regions and/or structural elements may be reduced or exaggerated for clarity. The use of similar or identical reference numbers in the various drawings is intended to indicate the presence of a similar or identical element or feature.

### DETAILED DESCRIPTION

Example embodiments of the inventive concepts will now be described more fully with reference to the accompanying drawings, in which example embodiments are shown. Example embodiments of the inventive concepts may, however, be embodied in many different forms and should not be construed as being limited to the embodiments set forth herein; rather, these embodiments are provided so that this disclosure will be thorough and complete, and will fully convey the concept of example embodiments to those of ordinary skill in the art. In the drawings, the thicknesses of layers and regions are exaggerated for clarity. Like reference numerals in the drawings denote like elements, and thus their description will be omitted.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Like numbers indicate like elements throughout. As used herein the term “and/or” includes any and all combinations of one or more of the associated listed items.

Other words used to describe the relationship between elements or layers should be interpreted in a like fashion (e.g., “between” versus “directly between,” “adjacent” versus “directly adjacent,” “on” versus “directly on”).

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises”, “comprising”, “includes” and/or “including,” if used herein, specify the presence of stated features, integers, steps, operations, elements and/or components, but do not preclude the presence or addition of one or more other features, integers, steps, operations, elements, components and/or groups thereof.

Example embodiments of the inventive concepts are described herein with reference to cross-sectional illustrations that are schematic illustrations of idealized embodiments (and intermediate structures) of example embodiments. As such, variations from the shapes of the illustrations as a result, for example, of manufacturing techniques and/or tolerances, are to be expected. Thus, example embodiments of the inventive concepts should not be construed as limited to the particular shapes of regions illustrated herein but are to include deviations in shapes that result, for example, from manufacturing. For example, an implanted region illustrated as a rectangle may have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of a device and are not intended to limit the scope of example embodiments.

Unless otherwise defined, all terms (including technical and scientific terms) used herein have the same meaning as commonly understood by one of ordinary skill in the art to which example embodiments of the inventive concepts belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

According to example embodiments of the inventive concepts, the chitosan spreading system may be configured to biologically treat a surface or skin of a biological object, as will be described with reference to the accompanying drawings.

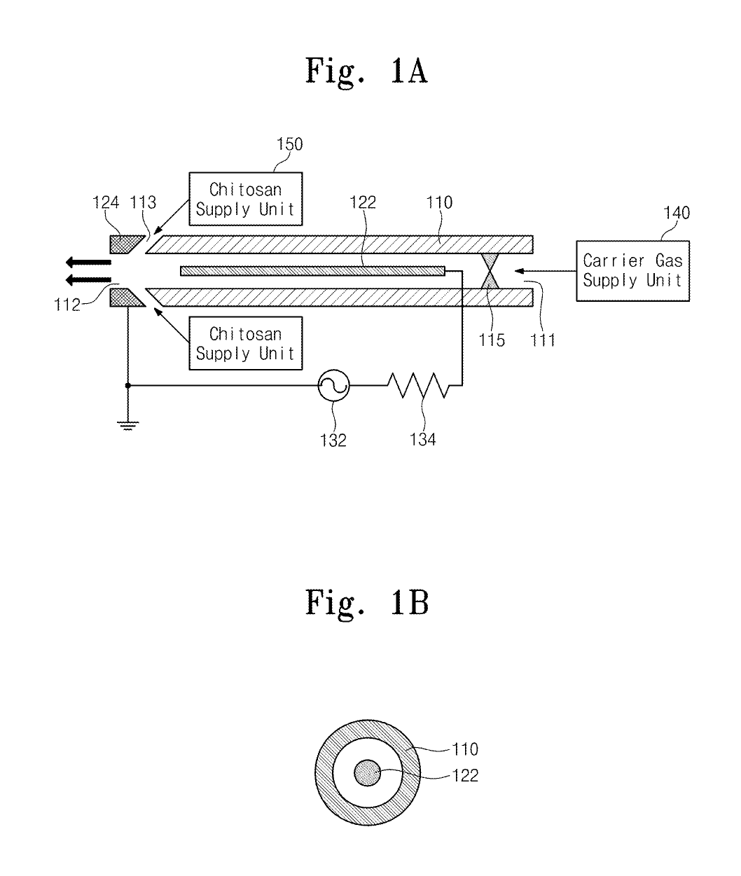

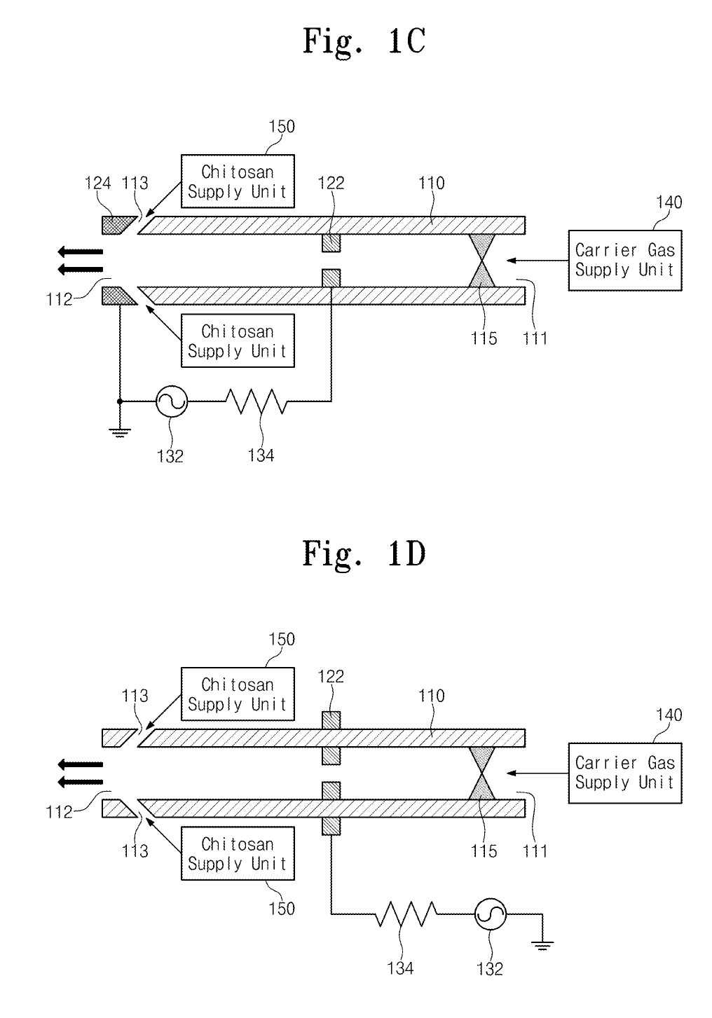

FIGS. 1A through 1D are sectional views illustrating a chitosan spreading system according to example embodiments of the inventive concepts.

Referring to FIGS. 1A through 1D, the chitosan spreading system may include a dielectric tube 110, a power electrode 122, a ground electrode 124, an power supply unit 132, a carrier gas supply unit 140, and a chitosan supply unit 150.

The dielectric tube 110 may be formed to have a hollow cylindrical shape with an internal empty space, and be formed of a dielectric material, such as quartz or alumina The dielectric tube 110 may have a gas inlet 111 connected to the carrier gas supply unit 140 and a plasma outlet 112 configured to spray plasma. The gas inlet 111 and the plasma outlet 112 may be formed spaced apart from each other. In some embodiments, the gas inlet 111 and the plasma outlet 112 may have the substantially same diameter as each other. According to the present embodiments, a chitosan inlet 113 may be formed on a sidewall of the dielectric tube 110, and the chitosan inlet 113 may be connected to the chitosan supply unit 150.

The power electrode 122 may be disposed within an internal space of the hollow-cylindrical dielectric tube 110. In some embodiments, the power electrode 122 may be shaped like a pin or a rod as shown in FIGS. 1A and 1B. The power electrode 122 may be coated with a dielectric material. In other embodiments, the power electrode 122 may be disposed in the dielectric tube 110 to have a sectional shape resembling the dielectric tube 110; for instance, the power electrode 122 may be shaped like a ring or a tube, as shown in FIG. 1C.

The ground electrode 124 may be installed at one end portion of the dielectric tube 110 to serve as a nozzle for spraying plasma. In some embodiments, the ground electrode 124 may have a sectional shape (e.g., a ring or a tube) resembling the dielectric tube 110 and be coated with a dielectric material.

In other embodiments, as shown in FIG. 1D, the chitosan spreading system may not have the additional ground electrode 124, while it include the power electrode 122 disposed near the dielectric tube 110.

The power electrode 122 and the ground electrode 124 may be formed of at least one of copper, copper alloys, aluminum, aluminum alloys, or stainless steel alloys. In some embodiments, the power electrode 122 and the ground electrode 124 may be formed of at least one of tungsten, molybdenum, zirconium, tantalum, alloys thereof, or compounds thereof.

A power supply unit 132 may be disposed to connect the power electrode 122 with the ground electrode 124. The power supply unit 132 may be an electric power generator configured to generate pulsed direct current or alternating current. The power supply unit 132 may be configured to supply a radio frequency electric power of several tens to several hundreds of kHz to the power electrode 122 and/or the ground electrode 124. In some embodiments, a resistor 134 may be disposed between the power supply unit 132 and the power electrode 122 to prevent an arc discharge from occurring.

The carrier gas supply unit 140 may be configured to supply a carrier gas, such as nitrogen, oxygen, helium, argon, or carbon dioxide, into the dielectric tube 110.

A control valve 115 may be installed within the dielectric tube 110 to control a flow rate of the carrier gas. In some embodiments, the control valve 115 may be disposed near the gas inlet 111. Plasma plume may be controlled by adjusting a flow rate or amount of the carrier gas to be supplied into the dielectric tube 110. Furthermore, the plasma plume may be controlled by adjusting at least one of voltage, frequency, pulse period, and/or pulse duration.

The chitosan supply unit 150 may be an injection device configured to spread chitosan in an aerosol form. In some embodiments, the chitosan supply unit 150 may be configured to spread chitosan in an aerosol or particle form into the dielectric tube 110.

In the chitosan spreading system, an electric potential difference between the power electrode 122 and the ground electrode 124 may ionize the carrier gas injected by the carrier gas supply unit 140 to generate plasma of the carried gas. In some embodiments, the plasma may be in low temperature (e.g., 60° C. or less) and atmospheric pressure. The plasma generated in dielectric tube 110 may be sprayed to the outside at high speed via the nozzle. According to example embodiments of the inventive concepts, the chitosan supplied via the chitosan inlet 113 may be injected to the outside along with the plasma of the carried gas. In some embodiments, the nozzle may be disposed in such a way that a skin of biological object can be coated with the chitosan sprayed along with the plasma of the carried gas.

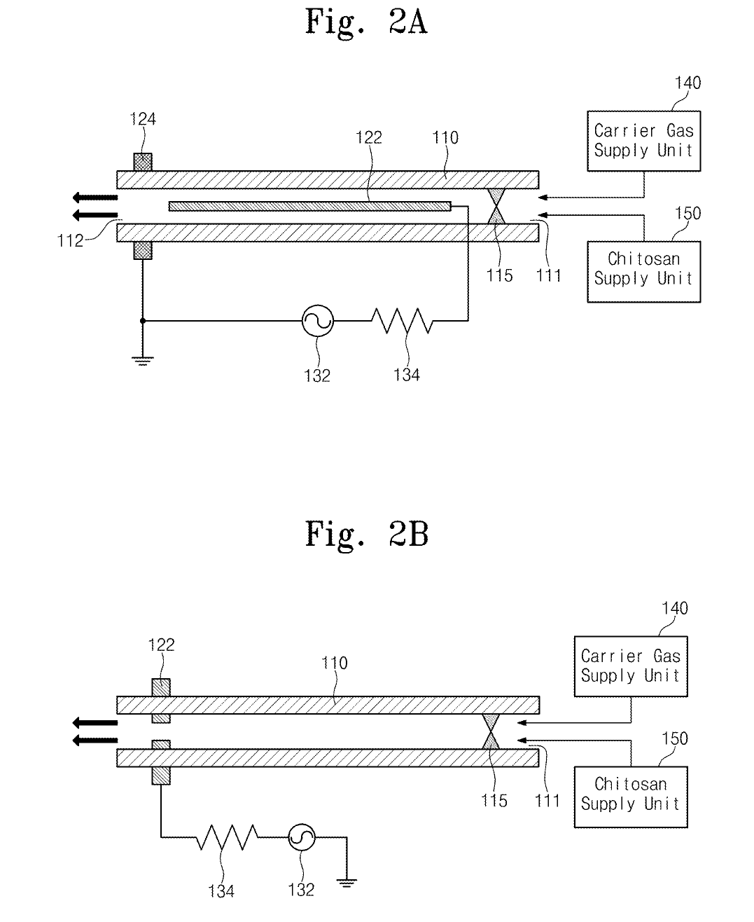

FIGS. 2A and 2B are sectional views illustrating a chitosan spreading system according to other example embodiments of the inventive concepts. For concise description, overlapping description of elements previously described with reference to FIGS. 1A through 1D may be omitted.

Referring to FIGS. 2A and 2B, the chitosan spreading system may include a dielectric tube 110, a power electrode 122, a ground electrode 124, a power supply unit 132, a carrier gas supply unit 140, and a chitosan supply unit 150.

According to the present embodiments, a carrier gas and chitosan may be supplied into the dielectric tube 110 via a gas inlet 111 provided at one end portion of the dielectric tube 110. In this sense, these embodiments differ from the previous embodiments of FIGS. 1A through 1D, in which the carrier gas and chitosan are independently supplied via the gas inlet 111 and the chitosan inlet 113. That is, according to the present embodiments, one end portion of the dielectric tube 110 may serve as the gas inlet 111 (to which the carrier gas and the chitosan may be supplied) and the other end portion thereof may serve as a plasma outlet 112. The carrier gas supply unit 140 and the chitosan supply unit 150 may be connected to the gas inlet 111 of the dielectric tube 110.

The power electrode 122 and the ground electrode 124 may be provided in the dielectric tube 110. In some embodiments, the power electrode 122 and the ground electrode 124 may be shaped like a rod and a tube, respectively, as shown in FIG. 2A. In other embodiments, as shown in FIG. 2B, the chitosan spreading system may not have the additional ground electrode 124, while it include the power electrode 122 disposed near the dielectric tube 110.

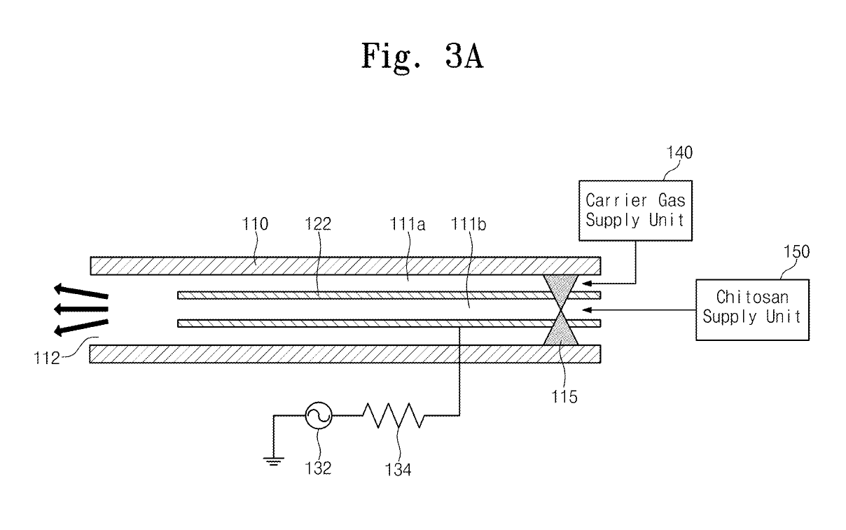

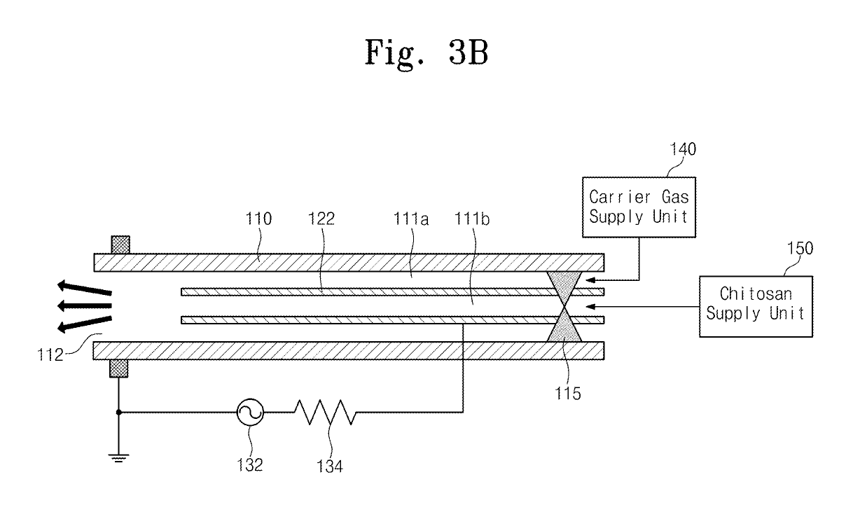

FIGS. 3A and 3B are sectional views illustrating a chitosan spreading system according to still other example embodiments of the inventive concepts. For concise description, overlapping description of elements previously described with reference to FIGS. 1A through 1D may be omitted.

Referring to FIGS. 3A and 3B, the chitosan spreading system may include a dielectric tube 110, a power electrode 122, a power supply unit 132, a carrier gas supply unit 140, and a chitosan supply unit 150.

The dielectric tube 110 may be formed to have a hollow cylindrical shape and be formed of a dielectric material, such as quartz or alumina.

The power electrode 122 may be formed to have a hollow cylindrical shape, and length and diameter thereof may be smaller than those of the dielectric tube 110. The power electrode 122 may be formed of a metal material and be coated with a dielectric. The power electrode 122 may be inserted into the dielectric tube 110. In some embodiments, chitosan may be supplied into the power electrode 122 via the power electrode 122. A carrier gas may be supplied into an internal region 111a interposed between the power electrode 122 and the dielectric tube 110.

The chitosan spreading system may further include a ground electrode 124 disposed on an outer surface of the dielectric tube 110, as shown in FIG. 3B. The ground electrode 124 may be formed to enclose the dielectric tube 110. For instance, the ground electrode 124 may have a ring or tube shape.

In addition, the chitosan spreading system may include a control valve 115 disposed near the dielectric tube 110 and/or the power electrode 122. The control valve 115 may be configured to control flow rates of the carrier gas and/or the chitosan.

According to the present embodiments, chitosan may be supplied via an internal space 111b of the power electrode 122, and then be mixed with low temperature atmospheric pressure plasma in a region of the dielectric tube 110 adjacent to the plasma outlet 112.

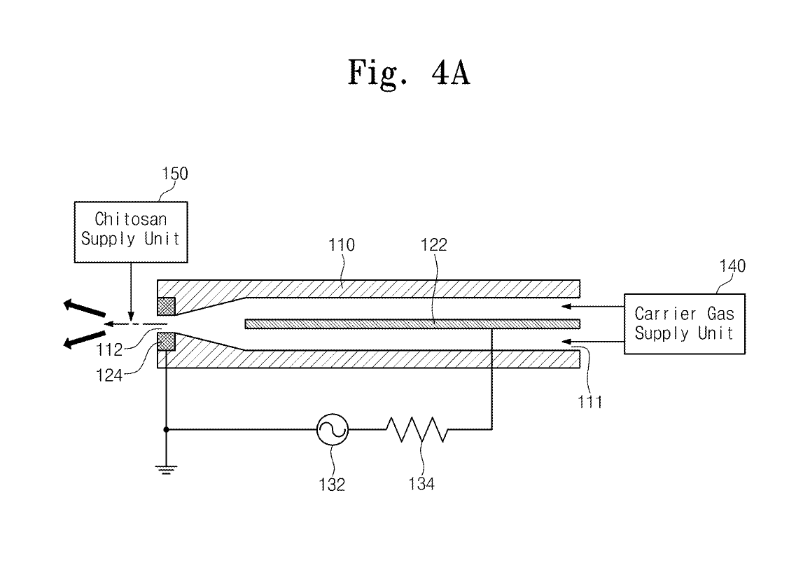

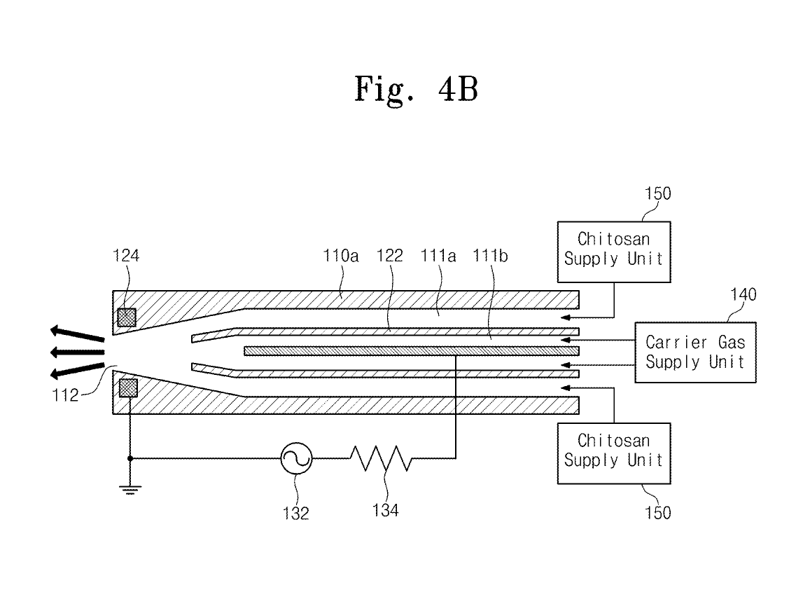

FIGS. 4A and 4B are sectional views illustrating a chitosan spreading system according to even other example embodiments of the inventive concepts. For concise description, overlapping description of elements previously described with reference to FIGS. 1A through 1D may be omitted.

According to the embodiments of FIG. 4A, the chitosan spreading system may include a dielectric tube 110, a power electrode 122, a ground electrode 124, an power supply unit 132, a carrier gas supply unit 140, and a chitosan supply unit 150.

The dielectric tube 110 may be formed to have a hollow cylindrical shape, and be formed of a dielectric material, such as quartz or alumina. The dielectric tube 110 may have a gas inlet 111 and a plasma outlet 112 provided at both end portions thereof In some embodiments, a diameter of the plasma outlet 112 may be smaller than that of the gas inlet 111.

In some embodiments, as shown in FIG. 4A, a carrier gas may be supplied via an internal space of the dielectric tube 110, and low temperature atmospheric pressure plasma may be injected to the outside through the plasma outlet 112 of the dielectric tube 110. Chitosan may be mixed with low temperature atmospheric pressure plasma at the outer region of the dielectric tube 110

In some embodiments, as shown in FIG. FIG. 4B, the chitosan spreading system may include first and second dielectric tubes 110a and 110b. The first dielectric tube 110a may have a gas inlet 111 and a plasma outlet 112 whose diameters are different from each other. The second dielectric tube 110b may be provided in the first dielectric tube 110a and be formed to have a hollow cylindrical shape. Length and diameter of the second dielectric tube 110b may be smaller than those of the first dielectric tube 110a.

The power electrode 122 may be provided in the second dielectric tube 110b. The power electrode 122 may be shaped like a pin or a rod. Alternatively, as shown in FIG. 1C, the power electrode 122 may be shaped like a ring or a tube in the dielectric tube 110. The ground electrode 124 may be provided at one end portion of the dielectric tube 110 to serve as a nozzle for spraying plasma.

According to the present embodiments, the chitosan spreading system may include the first and second dielectric tubes 110a and 110b, and the carrier gas may be supplied via an internal space of the second dielectric tube 110b provided with the power electrode 122. Chitosan may be supplied via a space confined by the first dielectric tube 110a and the second dielectric tube 110b.

Although not depicted, a control valve may be provided at other end portion of the dielectric tube 110, where the carrier gas and/or the chitosan may be supplied, as described with reference to FIGS. 3A and 3B.

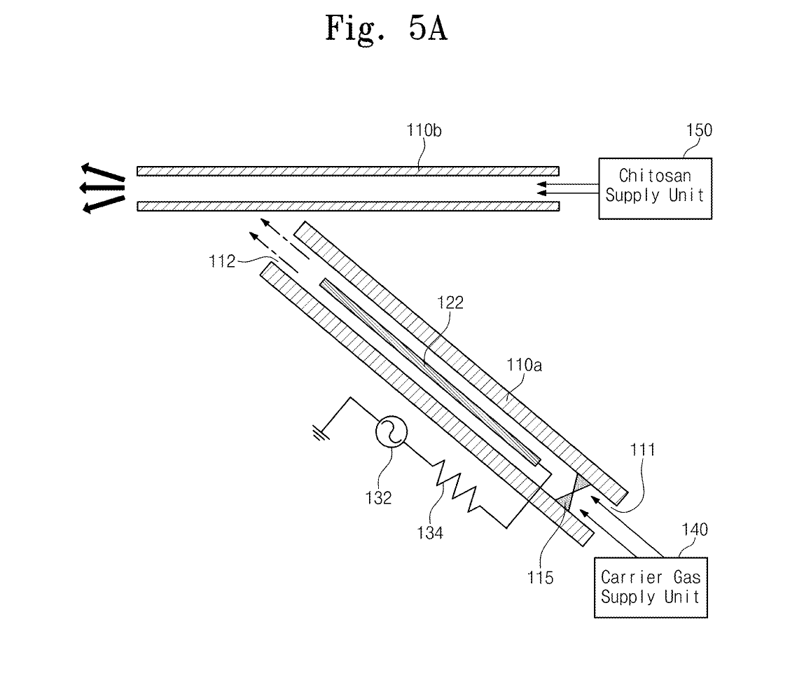

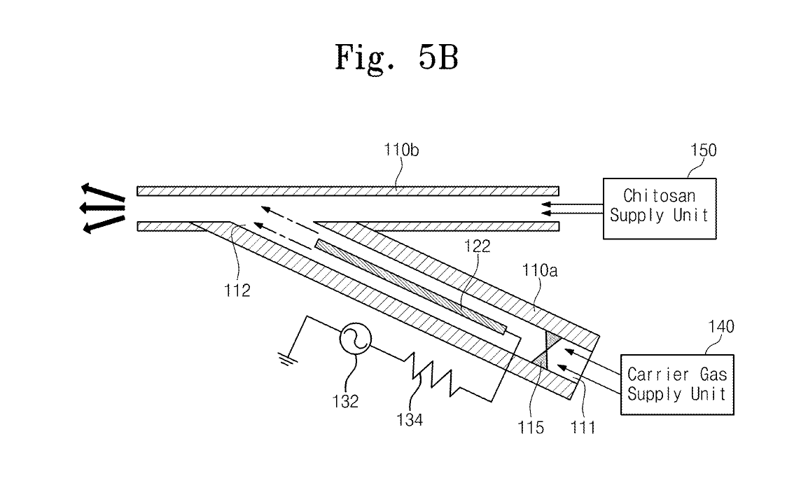

FIGS. 5A and 5B are sectional views illustrating a chitosan spreading system according to yet other example embodiments of the inventive concepts.

Referring to FIGS. 5A and 5B, the chitosan spreading system may include a plasma generating device and a chitosan supplying device. The plasma generating device may include a first dielectric tube 110a, a power electrode 122, an power supply unit 132, and a carrier gas supply unit 140. The chitosan supplying device may include a second dielectric tube 110b and a chitosan supply unit 150.

In the plasma generating device, the first dielectric tube 110a may be formed to have a hollow cylindrical shape connecting a gas inlet 111 and a plasma outlet 112. The power electrode 122 may be provided in the first dielectric tube 110a to have at least one of pin, rod, and tube shapes. In some embodiments, a ground electrode (not shown) may be provided at one end portion of the first dielectric tube 110a.

An electric power applied to the power electrode 122 may ionize the carrier gas injected into the first dielectric tube 110a via the carrier gas supply unit 140 to generate low temperature atmospheric pressure plasma. A flow rate or a flow amount of the carrier gas may be controlled by a control valve 115, and this control may be used to control plasma plume.

In some embodiments, the second dielectric tube 110b may be formed to have a hollow cylindrical shape, and chitosan may be injected via an internal space of the second dielectric tube 110b.

As shown in FIG. 5A, plasma plume excited in the plasma generating device may be supplied onto a sidewall of the second dielectric tube 110b of the chitosan supplying device to induce plasma in the second dielectric tube 110b delivering chitosan. In other words, the plasma plume generated in the plasma generating device may be used to generate plasma in the second dielectric tube 110b. As a result, plasma generated in the second dielectric tube 110b may have a lower temperature than that of plasma generated in the first dielectric tube 110a.

In some embodiments, as shown in FIG. 5B, the first dielectric tube 110a of the plasma generating device may be connected to the second dielectric tube 110b of the chitosan supplying device. For instance, the low temperature atmospheric pressure plasma generated in the plasma generating device may be injected into the second dielectric tube 110b and then be mixed with the chitosan passing through the second dielectric tube 110b. The mixture of chitosan and plasma may be sprayed onto a skin of a biological object.

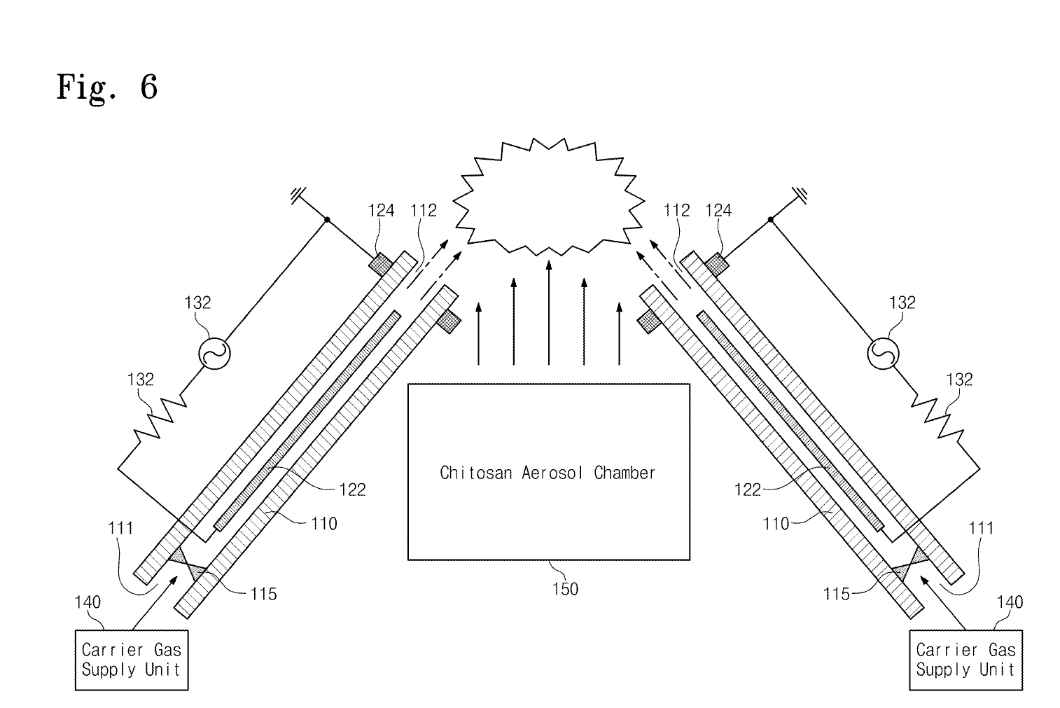

FIG. 6 is a sectional view illustrating a chitosan spreading system according to further example embodiments of the inventive concepts.

Referring to FIG. 6, the chitosan spreading system may include a plurality of plasma generating devices and a chitosan aerosol chamber.

Each of the plasma generating devices may include a dielectric tube 110, a power electrode 122, a ground electrode 124, and a carrier gas supply unit 140 and be configured to generate low temperature atmospheric pressure plasma. The chitosan aerosol chamber 150 may be configured to spread aerosol-type chitosan into a predetermined spraying region, and the plasma generating devices may be configured to supply low temperature atmospheric pressure plasma into the predetermined spraying region.

The chitosan sprayed from the chitosan aerosol chamber 150, along with the low temperature atmospheric pressure plasma, may be coated onto a skin of a biological object. This results from is because the low temperature atmospheric pressure plasma may be effective in improving skin surface properties and the chitosan may be effective in terms of blood clotting, sterilization and cell regeneration. As the synergistic effect of them, absorption and adhesion properties of chitosan on a skin can be improved.

According to example embodiments of the inventive concepts, a chitosan spreading system may be configured to spread chitosan on a skin of a biological object using low temperature atmospheric pressure plasma. This enables to improve absorption and adhesion properties of chitosan on a skin

In addition, biological properties of plasma (such as, blood coagulation, sterilization, or cell regeneration) can be synergically combined with medical properties of chitosan (such as, antibiosis, blood coagulation, and cell differentiation and growth). As a result, it is possible to expedite a skin wound healing process.

While example embodiments of the inventive concepts have been particularly shown and described, it will be understood by one of ordinary skill in the art that variations in form and detail may be made therein without departing from the spirit and scope of the attached claims.

What is claimed is:

1. A chitosan spreading system, comprising:
a dielectric tube of hollow cylindrical shape comprising a gas inlet supplied with a carrier gas and a plasma outlet spraying low temperature atmospheric pressure plasma generated therein;
a first electrode provided in the dielectric tube;
a power supply unit configured to apply an electric power to the first electrode;
a carrier gas supply unit configured to supply a carrier gas into the gas inlet of the dielectric tube; and
a chitosan supply unit configured to supply chitosan into the low temperature atmospheric pressure plasma generated in the dielectric tube.

2. The device of claim 1, wherein the dielectric tube further comprises a chitosan inlet provided on a sidewall of the dielectric tube and connected to the chitosan supply unit.

3. The device of claim 1, wherein the gas inlet of the dielectric tube is connected to both of the chitosan supply unit and the carrier gas supply unit, such that the carrier gas and the chitosan are supplied into the dielectric tube via the gas inlet.

4. The device of claim 1, wherein the chitosan supply unit is configured to mix the chitosan with the low temperature atmospheric pressure plasma sprayed from the plasma outlet.

5. The device of claim 1, wherein the first electrode has a rod shape or a ring shape.

6. The device of claim 1, wherein the first electrode has a substantially hollow cylindrical shape and length and diameter of the first electrode are smaller than those of the dielectric tube, and
the chitosan supply unit is configured to supply the chitosan into the first electrode, and the carrier gas supply unit is configured to supply the carrier gas into a region between the dielectric tube and the first electrode.

7. The device of claim 1, wherein a diameter of the plasma outlet is smaller than a diameter of the gas inlet.

8. The device of claim 1, further comprising a second electrode connected to the power supply unit, the second electrode being provided adjacent to the plasma outlet of the dielectric tube.

9. The device of claim 1, wherein the power supply unit further comprises a resistor connected to the first electrode in series to prevent an arc discharge from occurring.

10. A chitosan spreading system, comprising:
at least one plasma generating device comprising a first dielectric tube of hollow cylindrical shape comprising a gas inlet supplied with a carrier gas and a plasma outlet spraying low temperature atmospheric pressure plasma generated therein, a first electrode provided in the first dielectric tube, a power supply unit configured to apply an electric power to the first electrode, and a carrier gas supply unit configured to supply a carrier gas into the gas inlet of the first dielectric tube; and
a chitosan supplying device comprising a second dielectric tube of hollow cylindrical shape configured to supply chitosan into the low temperature atmospheric pressure plasma generated in the first dielectric tube.

11. The device of claim 10, wherein the plasma generating device and the chitosan supplying device are provided spaced apart from each other, and the plasma generating device is configured to spray the plasma generated therein onto a sidewall of the second dielectric tube of the chitosan supplying device.

12. The device of claim 10, wherein the first dielectric tube of the plasma generating device is connected to a sidewall of the second dielectric tube of the chitosan supplying device, such that the low temperature atmospheric pressure plasma generated in the plasma generating device is injected into an internal region of the second dielectric tube.

13. The device of claim 10, wherein the at least one plasma generating device comprises a plurality of plasma generating devices provided in the chitosan spreading system.

14. The device of claim 10, wherein the chitosan supplying device is configured to spread the chitosan in an aerosol form.
